(19) United States
(12) Patent Application Publication
(10) Pub. No.: US 2017/0281396 A1
(43) Pub. Date: Oct. 5, 2017

(54) PENILE VEIN-PRESSING ERECTION PROMOTER

(71) Applicants: Genglong Hsu, Taipei City (TW); Michael chihyuan Hsu, Taipei City (TW)

(72) Inventors: Genglong Hsu, Taipei City (TW); Michael chihyuan Hsu, Taipei City (TW)

(21) Appl. No.: 15/508,111

(22) PCT Filed: Sep. 25, 2014
(86) PCT No.: PCT/CN2014/087392

§ 371 Date: Mar. 2, 2017

(51) Int. Cl. A61F 5/41

(52) U.S. Cl. CPC A61F 5/41; A61F 2005/414

(57) ABSTRACT

A penile vein-pressing erection promoter includes a main body, a sliding member, a first fixing member, a second fixing member, and a second ring portion. The main body includes a first ring portion. The sliding member, the first fixing member, and the second fixing member are mounted to the main body. The second ring portion is connected to the second fixing member. The first ring portion is used to trap the glans retro-coronal sulcus and the sliding member is use to block returning blood of the glans sinusoids. The first fixing member and the second fixing member block returning blood of the penile cavernous sinusoids through the deep dorsal vein and the cavernosal veins of penis. The second ring portion is used to trap the penile base and thus enlarge the penile volume.

### (a) TECHNICAL FIELD OF THE INVENTION

The present invention relates to an erection promoter, and more particularly to a penile vein-pressing erection promoter.

### (b) DESCRIPTION OF THE PRIOR ART

Human penile anatomy, erectile function physiology and patho-physiology of erectile dysfunction have been widely studied for centuries. However penile tunical architecture, erection related venous system and strategy for either enhancing penile erection or treating erectile dysfunction may leave room for improvement thus far. The present inventor has conducted a series of study and achieves the following conclusions:
(1) Referring to FIG. 8, penile tunica albuginea is a bi-layered structure with a 360° complete inner circular layer and a 300° incomplete outer longitudinal coat which is a continuation of ischiocavernosus and bulbospongiosus muscles.
(2) The dorsum of the tunica albuginea is gradually changed from convex, flat to concave shape along the retro-coronus, pendulous portion to penile base caudally.
(2) Referring to FIG. 9, between the tunica albuginea and Buck' s fascia, the erection related veins includes one deep dorsal vein, two cavernosal veins, and four para-arterial veins, not just one deep dorsal vein as conventionally acknowledged, they all distribute along the dorsal side of the penis and merge near the penile hilum whereby drain into body circulation. The erectile function of the males is generally severely affected by penile arterial insufficiency and veno-occlusive dysfunction. Persistent premature ejaculation is the most commonly known nightmare. Man who suffers from these diseases is likely prevented from family harmony. Although penile implant is a final golden solution for erectile dysfunction, this procedure inevitably causes damage to corpora carvernosa and may risk the penis with infection and even prosthesis loss. Based on protracted basic medicine and clinic research, the present inventor has proven the globally unique penile venous stripping surgery for treatment of penile veno-occlusive dysfunction. However, such surgical technique may be difficult to be popular because it is extremely challenging.

Thus, it would be more practical to all men if a device can be available, which is penis vein compression erection-assisting device. It is wearable externally to a flaccid penis which is capable to fulfill intromission, then the crutched penis can reach erection via penile arterial perfusion and venous compression resulting from vaginal wall stimulation. Furthermore the penile girth can be enhanced by adding gasket. Consequently multiple problems can be solved simultaneously including erectile dysfunction, premature ejaculation, and penis maldevelopment. A safe sex can be secured if a condom is added.

Thus, based on the idea of innovation and also based on decades' professional and practical experiences in penile reconstruction, i.e. the present inventor has made the present invention based on chronologically penile research, numerous hemodynamic experiments and engineering designs

### SUMMARY OF THE INVENTION

An objective of the present invention is to provide a penile vein-pressing erection promoter that enables a male to conduct penile coitus despite he suffers from penile vascular dysfunction, penis maldevelopment, and premature ejaculation. This invention is so tally with the penile anatomic structure that the device is not only to be safe but also to be easily operated.

To achieve the above objective, the solution adopted in the present invention is as follows:

A penile vein-pressing erection promoter comprises a main body, which comprises a bendable bar bent to form two support sections and a first ring portion connected to an end of the support sections, the first ring portion being provided, externally thereof, with a silicone jacket; a sliding member, which is slidably fit to the support sections of the main body; a first fixing member, which is mounted to the support sections of the main body; a second fixing member, which is mounted to an opposite end of the support sections of the main body; and a second ring portion, which is connected to the second fixing member, the second ring portion being provided, externally thereof, with a second ring portion the silicone jacket.

The main body comprises a steel/ wire.

The first ring portion comprises an oval ring.

The first ring portion inclines downward by forty-five degrees from an end of the support sections.

The second ring portion comprises an oval ring.

The sliding member comprises a flat planar surface formed on an upper side thereof and a concave surface on a lower side thereof The concave surface is engageable with a surface of a penis.

The first fixing member comprises a flat planar surface formed on an upper side thereof and a convex surface on a lower side thereof. The convex surface is engageable with a surface of a penis.

The first fixing member comprises a flat planar surface formed on an upper side thereof and a convex surface on a lower side thereof. The convex surface is engageable with a surface of a penis.

A reinforcement member is arranged between the first fixing member and the second fixing member.

The second ring silicone jacket is arranged to partly cover the second ring portion to form two recesses. Two lumbar fastener belts are respectively coupled to portions of the second ring portion in the recesses with one end thereof. The lumbar fastener belts re provided at an opposite end thereof with fastening sections. The fastening sections are attachable to each other in a movable manner.

By adopting the above structure, the penile vein-pressing erection promoter of the present invention uses the first ring portion to trap the retro-coronal sulcus, the sliding member blocking returning blood of the glanular sinusoids, the first fixing member and the second fixing member blocking returning blood of the penile cavernous sinusoids through the deep dorsal vein and the cavernosal vein of penis, the second ring portion trapping the penile base and enlarging the penile shaft, and the support sections of the main body providing an effective supporter. The device can be entirely worn on the penile shaft of a male and then enable the crutched penis to do intromission, even not in a condition of erection, wearing being safe, so that through repeated stroke abrasion with vagina wall, stimulation is made to encourage penile arterial blood perfusion to the cavernous sinusoids and achieve bettered erection and penile volume expansion, allowing for more tight engagement between a condom and the penis body, also helping delay ejaculation time, benefiting males suffering from erection dysfunction or premature ejaculation, after wearing the device, enabling to do intromission so that, through stimulation, enhancing penile arterial blood to the cavernous sinusoids can be achieved, meanwhile the erection related draining venous system is blocked. Consequently a full erection might be achieved and thus could effectively treat a patient who suffers from penile vascular dysfunction or refractory premature ejaculation and enlarging the penile base.

### BRIEF DESCRIPTION OF THE DRAWINGS

FIG. 1 is a perspective view of the present invention.

FIG. 2 is a perspective view, partly exploded, of the present invention.

FIG. 3 is a perspective view of the present invention taken from a different angle.

FIG. 4 illustrates an example of application, demonstrating further expansion of a first ring according to the present invention.

FIG. 5 is a schematic view illustrating an application of the present invention.

FIG. 6 is a schematic view illustrating a structure of a first embodiment of the present invention.

FIG. 7 is a schematic view illustrating a structure of a second embodiment of the present invention.

FIG. 8 is a schematic view illustrating an anatomic structure of penile tunica albuginea.

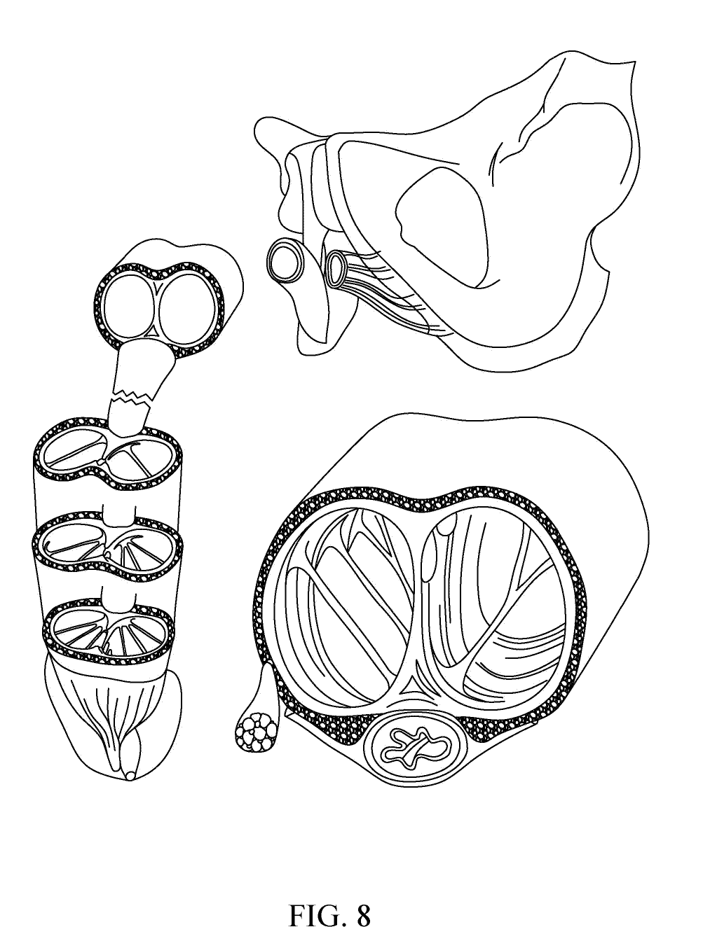

FIG. 9 is a diagram illustrating venous system related to penile erection.

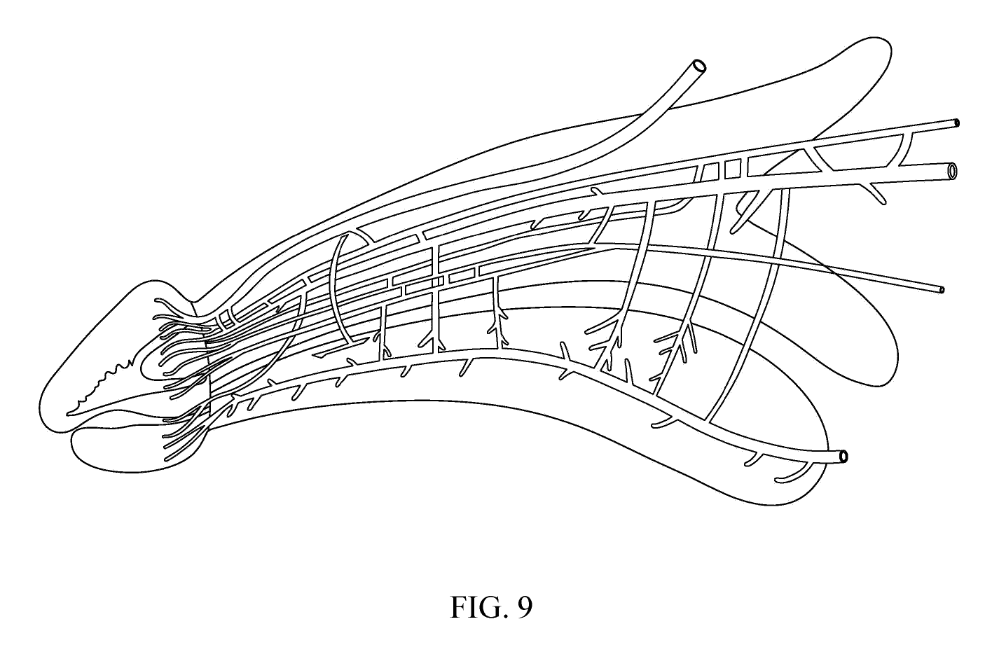

### DETAILED DESCRIPTION OF THE PREFERRED EMBODIMENTS

For better expound the technical solution of the present invention, a detailed description of the present invention is given below with reference to an embodiment thereof.

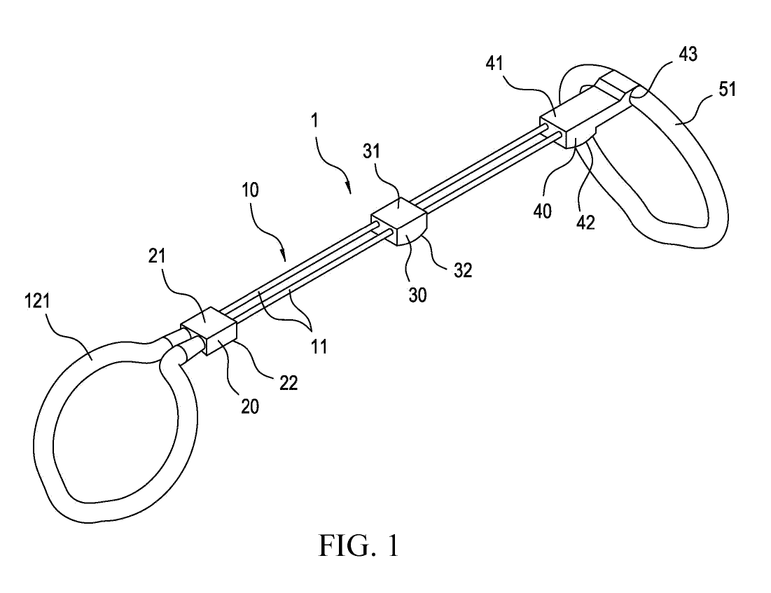

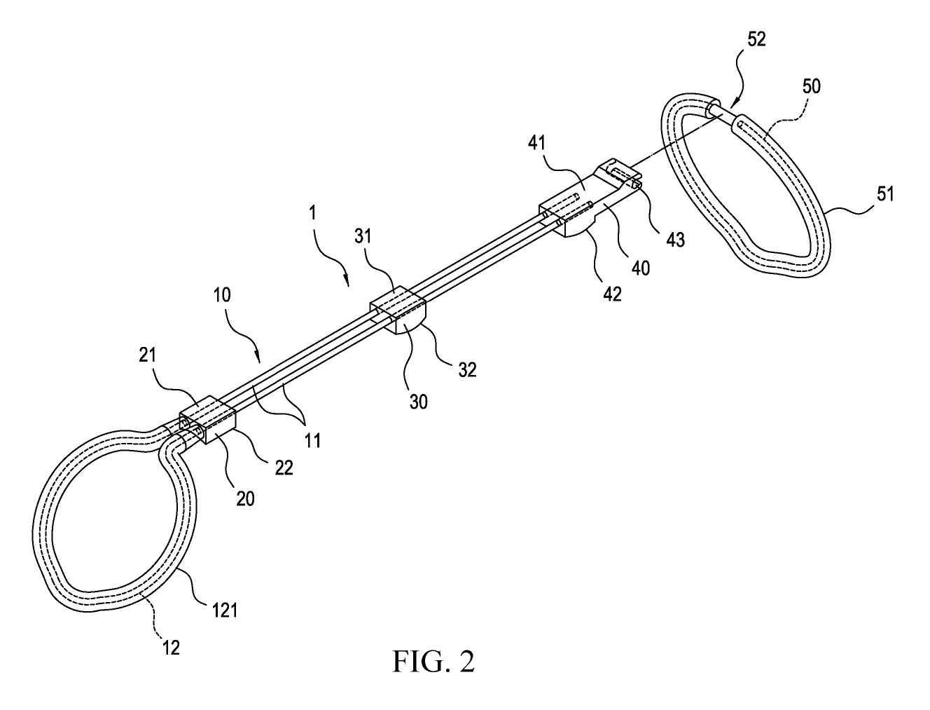

Referring to FIGS. 1 and 2, the present invention provides a penile vein-pressing erection promoter 1, of which a preferred embodiment comprises a main body 10, a sliding member 20, a first fixing member 30, a second fixing member 40, and a second ring portion 50.

The main body 10 comprises a bendable bar and is bent to form two support sections 11 and a first ring portion 12 connected to one end of the support sections 11. In the instant embodiment, the two support sections 11 constitute two parallel bar sections. The support sections 11 provide an effect of supporting. The first ring portion 12 is provided, externally thereof, with a silicone jacket 121. The first ring portion 12 is operable to trap the glans coronal sulcus, and the silicone jacket 121 helps improve comfortableness and safety of wearing and may serve as enlargement of the penile shaft. As such, the main body 10 is worn on the penile shaft of a male to support the penis at a length close to that in an erected condition and establish a desired hardness for fulfilling intromission.

The sliding member 20 is slidably fit to the support sections 11 of the main body 10. As such, the sliding member 20 is slidable to control the first ring portion 12 to open and close of a predetermined outside diameter for trapping the glans coronal sulcus therein.

The first fixing member 30 is mounted to the support sections 11 of the main body 10.

The second fixing member 40 is mounted to an opposite end of the support sections 11 of the main body 10.

The second ring portion 50 is connected to the second fixing member 40. The second ring portion 50 is provided, externally thereof, with a second ring silicone jacket 51. As such, the second ring portion 50 may trap the penile base, the second ring silicone jacket 51 helps improve comfortableness and safety of wearing. In the instant embodiment, the second ring portion 50 comprises a 3-centimeter wide silicone sleeve wrapping around the penile base to achieve enlargement of the penile shaft. Additional assistance can be achieved for those having a thin penile base for achieving a more tight engagement between a condoms and the penile body to provide better protection of safe sex.

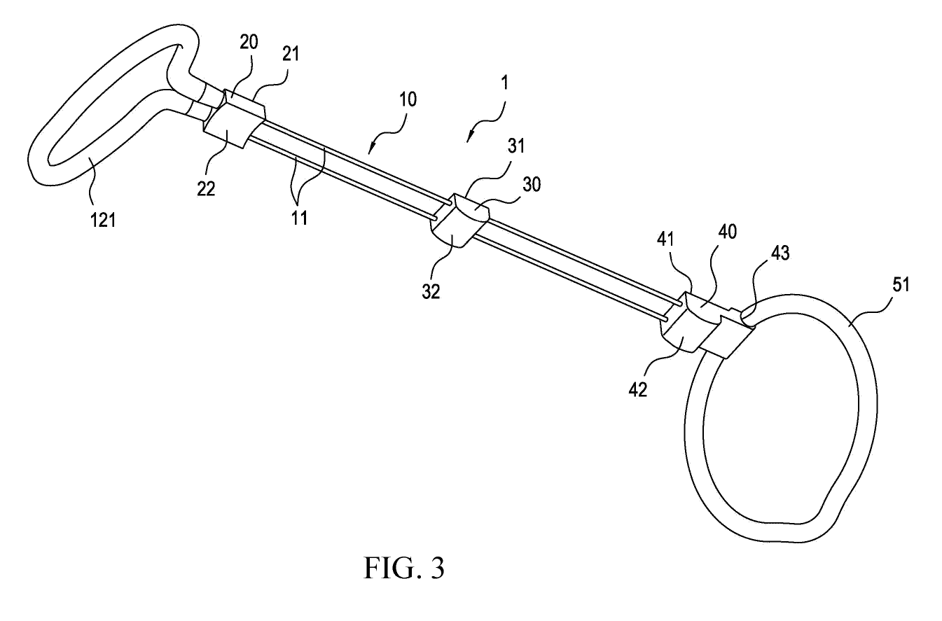

Referring to FIGS. 2 and 3, in the instant embodiment, the main body 10 comprises a steel wire and thus, provides an effect of supporting and also enhancing strength.

In the instant embodiment, the first ring portion 12 is of an oval shape and the first ring portion 12 inclines downward by 45 degrees from a front end of the support sections 11 so as to match the shape of the glans coronal sulcus for easy wearing. The oval ring prevent the urethra from ejaculation obstacle.

In the instant embodiment, the second ring portion 50 is oval in shape so as to match the shape of the penile base for easy wearing.

In the instant embodiment, the sliding member 20 is provided with a flat planar surface 21 on an upper side thereof and a concave surface 22 on a lower side thereof. As such, contact can be made with the surface of the penis with the concave surface 22.

In the instant embodiment, the first fixing member 30 is provided with a flat planar surface 31 on an upper side thereof and a convex surface 32 on a lower side thereof. As such, contact can be made with the surface of the penis with the convex surface 32.

In the instant embodiment, the first fixing member 40 is provided with a flat planar surface 41 on an upper side thereof and a convex surface 42 on a lower side thereof. As such, contact can be made with the surface of the penis with the convex surface 42.

In the instant embodiment, the second fixing member 40 comprises a coupling groove 43 formed in a lateral side thereof. The second ring silicone jacket 51 does not completely cover the second ring portion 50 so as to define a recess 52, allowing the coupling groove 43 to be fit to and fixed to the portion of the second ring portion 50 in the recess 52 so as to connect the second ring portion 50 to the second fixing member 40, but allowing for movable adjustment adequately.

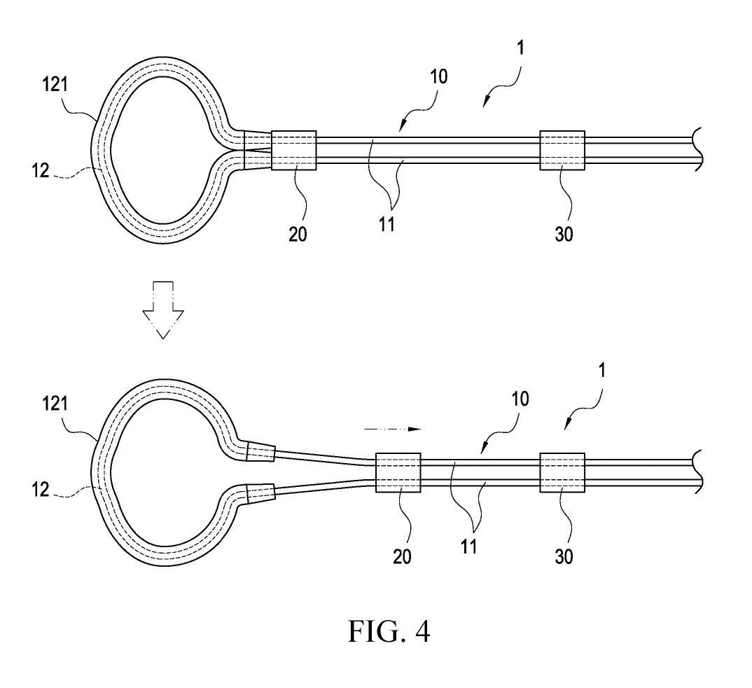

Referring to FIG. 4, to use, since the sliding member 20 is slidably fit to the support sections 11 of the main body 10, meaning being slidable on a front half of the penile shaft, when the sliding member 20 is moved backward, the support sections 11 is acted upon by a force to elastically expand outward, allowing the oval first ring portion 12 to wide open for trapping the glans coronal sulcus, while the sliding member 20 is fixed at the rear side of the glans coronal to prevent draining blood from the glans sinusoids into the veins.

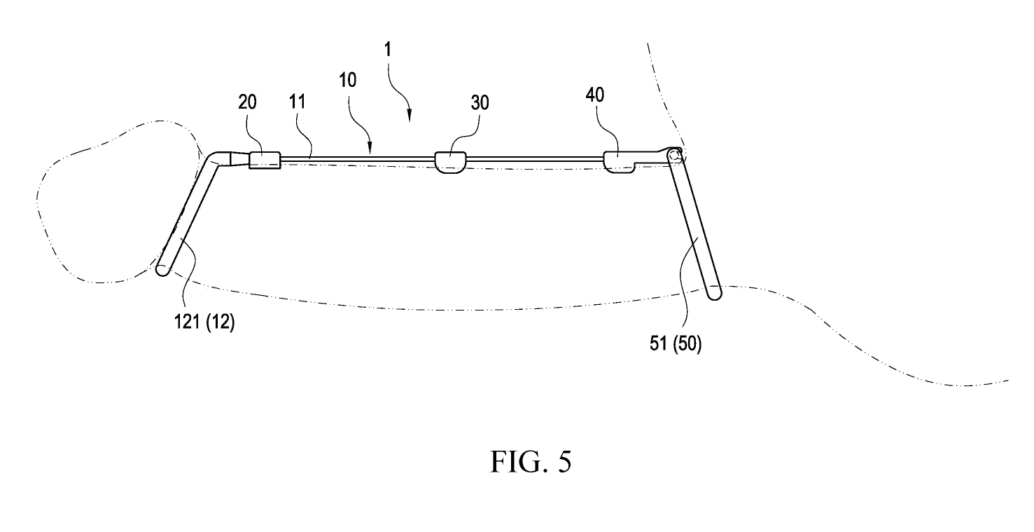

Referring to FIG. 5, the above provides an introduction to the components of the present invention. In the following, an operation of assembly, principle, and features of these components will be described.

To use the penile vein-pressing erection promoter 1 of the present invention, the first ring portion 12 is used to trap the glans retro-coronal sulcus and the sliding member 20 is use to block returning blood of the glans sinusoids. The first fixing member 30 and the second fixing member 40 block returning blood of the penile cavernous sinusoids through the deep dorsal vein and the cavernosa vein of penis. The second ring portion 50 is used to trap the penile base and thus enlarge the penile volume. The device can be entirely worn on the penile shaft of a male who suffers from erectile dysfunction or refractory premature ejaculation, after wearing the device, enabling to do intromission, so that, through stimulation, enhancing penile arterial blood to the cavernous sinusoids can be achieved, meanwhile the erection related draining venous system is blocked. Consequently it could treat a man who suffers penile vascular dysfunction or premature ejaculation and enlarges the penile base for man who suffers from penile maldevelopment.

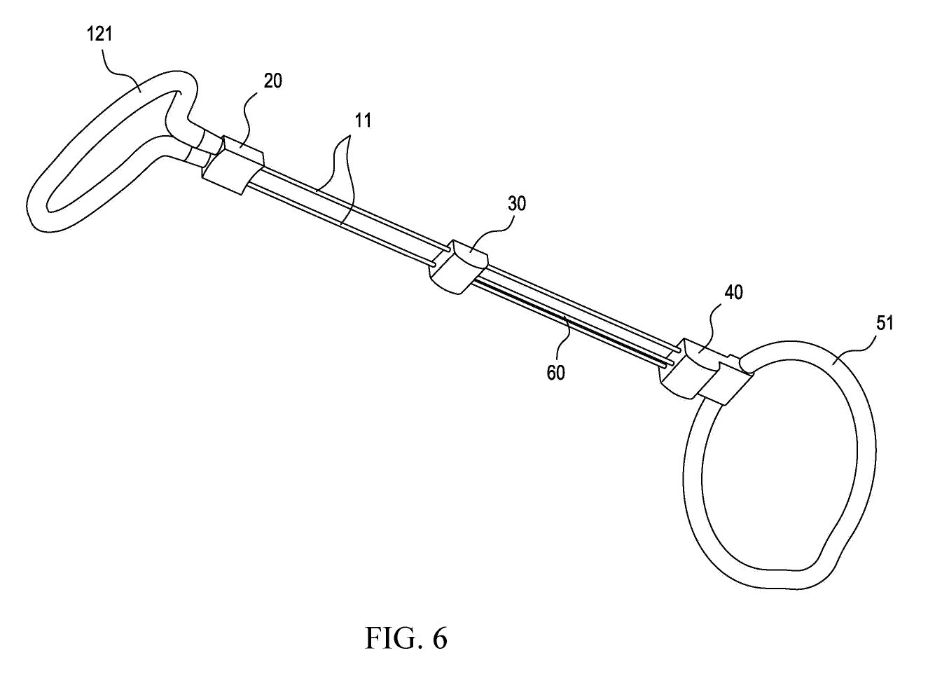

Referring to FIG. 6, in an embodiment, additionally provided between the first fixing member 30 and the second fixing member 40 is a reinforcement member 60 to reinforce the supporting strength.

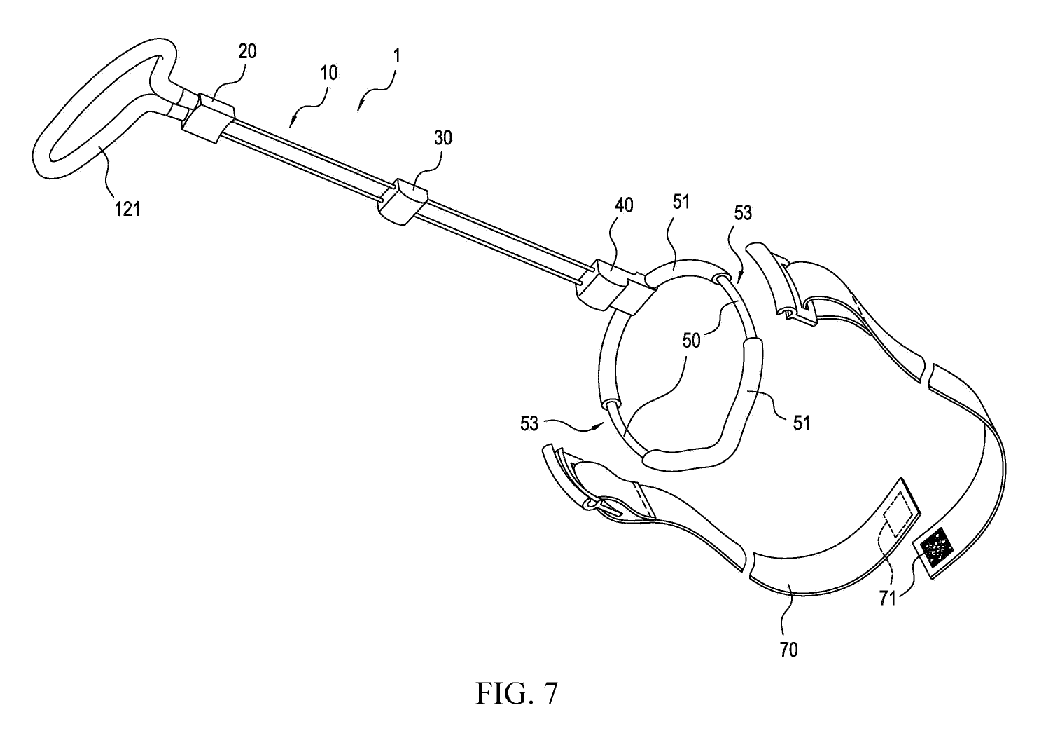

Referring to FIG. 7, in an embodiment, the second ring silicone jacket 51 does not completely cover the second ring portion 50 and form two recesses 53, allowing two lumbar fastener belts 70 to couple, at one end thereof, to the portions of the second ring portion 50 in the recesses 53. The lumbar fastener belts 70 are provided at an opposite end thereof with a fastening section 71, such as hook and loop fasteners. The fastening sections 71 may be attached to each other and as such, with the lumbar fastener belts 70 wrapped around and attached to the lower lumbar of a human body, stability of wearing of the present invention can be enhanced.

In summary, the present inventor made the penile vein-pressing erection promoter of the present invention by making a reference to a ring-shaped electric knife that is used in the transurethral resection of the prostate. Blood circulates back to the body from the glans penis and the cavernous sinusoids through the deep dorsal vein and the cavernosal veins of the penis. This new discovery makes it possible to completely handle the factors of sufficiently rigid erection and also provides the insight why a rigid erection can be achieved by having finger grapping the base of a semi-erected penis shaft as presented in an impotent male. Thus, by conforming to the convex of the outside appearance of the tunica algubinea, a sliding member, a first fixing member, and a second fixing member that effectively block venous returning blood are designed. The first ring portion that traps the glans sinusoidal blood therein is not in a circular shape, but instead, an oval shape for the purposes of ensuring smoothness of ejaculation. Overall, the present invention can treat a patient who suffers from erectile dysfunction to achieve penile rigidity and then negate the option of surgery. The present invention is a design conforming anatomic physiology, and is additionally assisted with the second ring silicone jacket to enlarge the penile shaft, delay ejaculation time, tightly engage a condom to secure safe sex and also treat erectile dysfunctions resulting from penile veno-occlusive dysfunction or penile arterial insufficiency and suffering from penis maldevelopment or refractory premature ejaculation. Such a structure has not been disclosed in any existing techniques and meets the patentability requirement of novelty and non-obviousness.

The embodiments and drawings described above are not provided to impose constraints to the product models and types of the present invention. Any suitable variation and modification made by those having ordinary skills in the art should be regarded as belonging to the scope of the present invention as defined in the appended claims.

I claim:

1. A penile vein-pressing erection promoter, comprising:
a main body, which comprises a bendable bar bent to form two support sections and a first ring portion connected to an end of the support sections, the first ring portion being provided, externally thereof, with a silicone jacket;
a sliding member, which is slidably fit to the support sections of the main body;
a first fixing member, which is mounted to the support sections of the main body;
a second fixing member, which is mounted to an opposite end of the support sections of the main body; and
a second ring portion, which is connected to the second fixing member, the second ring portion being provided, externally thereof, with a second ring portion the silicone jacket.

2. The penile vein-pressing erection promoter according to claim 1, wherein the main body comprises a steel wire.

3. The penile vein-pressing erection promoter according to claim 1, wherein the first ring portion comprises an oval ring.

4. The penile vein-pressing erection promoter according to claim 1, wherein the first ring portion inclines downward by forty-five degrees from an end of the support sections.

5. The penile vein-pressing erection promoter according to claim 1, wherein the second ring portion comprises an oval ring.

6. The penile vein-pressing erection promoter according to claim 1, wherein the sliding member comprises a flat planar surface formed on an upper side thereof and a concave surface on a lower side thereof, the concave surface being adapted to engage a surface of a penis.

7. The penile vein-pressing erection promoter according to claim 1, wherein the first fixing member comprises a flat planar surface formed on an upper side thereof and a convex surface on a lower side thereof, the convex surface being adapted to engage a surface of a penis.

8. The penile vein-pressing erection promoter according to claim 1, wherein the first fixing member comprises a flat planar surface formed on an upper side thereof and a convex surface on a lower side thereof, the convex surface being adapted to engage a surface of a penis.

9. The penile vein-pressing erection promoter according to claim 1, wherein a reinforcement member is arranged between the first fixing member and the second fixing member.

10. The penile vein-pressing erection promoter according to claim 1, wherein the second ring silicone jacket is arranged to partly cover the second ring portion to form two recesses, two lumbar fastener belts being respectively coupled to portions of the second ring portion in the recesses with one end thereof, the lumbar fastener belts being provided at an opposite end thereof with fastening sections, the fastening sections being attachable to each other in a movable manner.
